(19) United States
(12) Patent Application Publication
(10) Pub. No.: US 2025/0266156 A1
(43) Pub. Date: Aug. 21, 2025

(54) NURSE-TO-PATIENT ASSIGNMENT TOOL (NPAT)

(71) Applicants: KATHERINE ORTALEZA CARANDANG, Austin, TX (US); FELIPE EDSEL PADILLA CARANDANG, Austin, TX, TX (US); SIMON HADIAH ORTALEZA CARANDANG, Austin, TX (US); JACQUES MICAH ORTALEZA CARANDANG, Austin, TX (US)

(72) Inventors: KATHERINE ORTALEZA CARANDANG, Austin, TX (US); FELIPE EDSEL PADILLA CARANDANG, Austin, TX, TX (US); SIMON HADIAH ORTALEZA CARANDANG, Austin, TX (US); JACQUES MICAH ORTALEZA CARANDANG, Austin, TX (US)

(21) Appl. No.: 18/803,943

(22) Filed: Aug. 14, 2024

Related U.S. Application Data
(60) Provisional application No. 63/555,685, filed on Feb. 20, 2024.

(51) Int. Cl. G16H 40/20; G16H 15/00

(52) U.S. Cl. CPC G16H 40/20; G16H 15/00

(57) ABSTRACT

This invention is a tool in the form of a physical board or a computer software that displays each nurse's name followed by the patient room numbers assigned to him/her whereby the room number is circumscribed in a box color-coded by the patient's acuity—i.e., level of difficulty in administering required care—where red, yellow and green indicate high, moderate and low acuity, respectively. Along with these, other patient information such as hall/pod location, specialized treatment needs as well as the isolation precautions that nurses and patient-care technicians need to observe when attending to the patient are also shown in each box. This invention leverages color as a way of facilitating the distribution of patient assignment and enables display in clear view of anyone a bird's-eye view of the nurse-to-patient assignment, which greatly enhances nurses' perception of an unbiased and equitable distribution of their assignments.

### REFERENCE TO RELATED APPLICATIONS

Application Number: 63/555,685

### STATEMENT OF FEDERALLY SPONSORED RESEARCH

Not Applicable

### REFERENCE TO A SEQUENCE LISTING

“Non-Provisional_Utility_Patent_2024_4FIGURES.docx”

### FIELD OF THE INVENTION

Nursing and allied non-medical professions (e.g., occupational therapy, physical therapy) whose practice in the hospital setting requires attending and administering care to multiple patients in a work shift. For brevity, the term “nurses” heretofore is used to refer to “nurses, allied non-medical professionals and/or patient care technicians (PCT) who administer care to multiple patients in a work shift. By extension, therefore, the term “nursing” will be used to refer to “nursing and allied non-medical professions.”

### BACKGROUND OF THE INVENTION

American hospitals have communicated risks of limited resources in the productivity and efficiency of hospital organizations. Specifically, nurses and caregivers express anxieties over the effects of deficiency in headcount on work quality in terms of safety, care and time consumed with patients. To resolve disproportionate workloads, hospitals have studied means by which to eradicate non-productive activities and equalize unit activities among the staff through enhanced planning. The Charge Nurse (CN) is responsible in giving workload assignments, which are essentially the nurse-to-patient assignments, for the shift. Today however, studies suggest that workload assignments have not been given more attention even though they have a direct and critical impact on the activities of the nurses during a shift (Acar & Butt, 2016).

This invention is based on the inventor's personal experience in 2018 as a primary care nurse (PCN) in the Neurology unit of an acute care hospital in Central Texas, where she observed unequal distribution of high-acuity patients as being the norm rather than the exception. This nurse-inventor also confirmed that neither the unit nor any in the whole hospital facility applies the Evidence-Based Practice (EBP) of using a Patient Assessment Tool (PAT), which will heretofore be referred to as a Standardized Patient Acuity Assessment Tool (SPAAT), as an aid in distributing Nurse-to-Patient assignments.

According to Kidd, Grove, Kaiser, Swoboda and Taylor (2014), majority of nurses in the medical-surgical units trust their supervisors to provide equal workload so that they can give excellent care to their patients basing on sound assessments. However, Kidd et al (2014) reported that CNs and PCNs base their assessment on their personal judgement which are often ineffective. These studies taken together, therefore, suggest that a workload assignment that is far from being equally distributed hampers effective assessments that ultimately impedes attainment of excellent patient outcomes. Studies have also shown that inequitable distribution of difficult patients—i.e., those with high acuity levels-leads nurses into harboring feelings of restlessness, incompetence and frustration (Kidd, et al, 2014). Research has further shown that the use of a tool that objectively distributes assignments based on a standardized measure of patient acuity leads to a safe, fair and balanced distribution of challenging patients, (Chuilli, Thompson, & Reguin-Hartman, 2014; Jones, 2015). Concerned with the safety and well-being of both the patient, on one hand, and the nurses, on the other, this inventor proposed on Dec. 17, 2017 a quality improvement project for aforementioned Neurology unit using a SPAAT titled, “Promoting Equitable Nurse-to-Patient Assignments”.

The clinical study commenced on June 2018 aimed towards achieving an equitable—i.e. approximately proportional—distribution of Nurse-to-Patient assignment that is safe for both nurse and patients. The pre-intervention test (pre-test) showed 38% of the nurses were somewhat satisfied with the distribution of patient acuity in their daily assignment and 4% were completely satisfied. Following are some of the unedited feedbacks from nurses gathered from the pre-test study to wit:

“Having 3 total care patients out of 5 for 1 nurse is too much.”

“Patients that require frequent administration also require a lot of attention and should be considered like a total (care).”

“Pain meds-Q2, Q3, Q41; total care/bedfast; multiple drains; admissions; giving blood; isolation; Altered Mental Status”1 The terms “Q2”, “Q3” and “Q4” mean “every two hours”, “every three hours” and “every four hours”, respectively.

“Sometimes you will have 4-5 isolation patients to a single nurse.”

“Distribute the isolation patient to different RNs (Registered Nurse). It is time consuming, try to assign the patients to a nurse in a single pod or at least to nearby pods as possible; make less nurses for hand-off; sometimes we have to give report to 6 different nurses.”

“Giving 1 nurse 2 blood transfusions.”

“Giving 1 nurse 5 out of 6 total care patients.”

“Giving 1 nurse 3-4 isolation patients.”

“Giving 1 nurse TPN² patient and tube feed patient.”2 Total Parenteral Nutrition

“Giving 1 nurse more than 1 PICC³ line to do blood draws on.”3 Peripherally Inserted Central Catheter

“Sometimes we have 4 total care patients from total of 6 with high acuity and assignment”

“ . . . is in 3 different pods with bed alarms and Avasys.”

“They used to ask each nurse what the acuities of each patient are—I don't see that now; Older/more experienced nurses get the harder, sicker (sic) patients; nicer nurse will get the more demanding, hard patient; not favoritism, it's more on who can handle the demanding patients.”

“Stronger nurses tend to get a heavier load, whereas the others are given lighter loads.”

“A few weeks ago, all 5 of my patients were on-isolation. This happens rarely but I thought this wasn't fair at the time when other nurses had none.”

“Some nurses will get easy patients and we can see them sitting, searching websites; some nurses will be running without taking breaks, not even doing charting or anything; all total care to 1 nurse or most contact precautions, some nurses will not get any total and contacts.”

“Unable to assist patient to the bathroom in a timely manner due to nurse and tech changing total care patient.”

Based on the various feedback received, a SPAAT appropriate for the patient population of the unit was created and used. Subsequently, each patient's room number and hall/pod location, specialized treatment needs and isolation precautions (to be observed by the nurse assigned) were identified; and an overall SPAAT-based acuity level assigned. All these patient information were posted on the first version of this invention, which is a physical board, thus putting it to task on its first clinical trial.

### Results of Clinical Trial

The post-intervention test (post-test) showed that 50% of the nurses were somewhat satisfied and 25% were completely satisfied. Additional survey showed that 92% of the nurses thought that the use of the NPAT resulted in a more equitable distribution of the workload. Table 1 summarizes the results of this clinical test that showed an increase in satisfaction from the pre-test.

TABLE 1
Summary of Clinical Test Results

| | Pre-Test | Post-Test | Increase |
|---|---|---|---|
| Somewhat Satisfied | 38% | 50% | 12% |
| Completely Satisfied | 4% | 25% | 21% |

Below are some of the unedited post-test feedback from nurses about this invention, to wit:

“It is easier to make the assignment using the Nurse-to-Patient assignment tool. It is easier to see the acuity at one glance. Eliminates the chance to assign duplicates (same room to different nurses) or missed any room that needs to be assigned.”-floated charge nurse

“The equitable distribution of patient assignments to nurse is an awesome project. It really helps balance the workload. Good job!”

“It is a great tool for accurate Nurse-to-Patient assignment.”

“Just by looking at the board tells me how many nurses I need to keep the unit safe, and I can easily convince management.”-Nurse Manager

### SUMMARY OF THE INVENTION

This invention is called the “Nurse-to-Patient Assignment Tool” (NPAT). It is a tool that
1) facilitates assessment of patients based on the EBP of using a SPAAT, 2) utilizes the output of the SPAAT as input to the NPAT, and 3) facilitates an unbiased and equitable distribution of patient assignments across nurses on-shift through a color-coded, bird's-eye-view and easily adjustable display of patient information.

This invention is available in two forms:
a physical board, or
a digital software.

### OBJECT OF THE INVENTION

Patients are admitted to the hospital to be treated until they are well. They do not expect their health to get any worse. Nurses, on the other hand, are tasked to help patients get better. They can do their job effectively in a manner that will ensure safety and enhance the well-being of their patients and their own when they are given a reasonable amount of workload. A reasonable workload can only be one that is based on an objective assessment of all patients and that is equitably distributed across all nurses and the like on-shift. Some hospital units may have been successful in maintaining an equitable workload distribution through some means. For those who are struggling to achieve this efficiently and effectively, this invention can be the leverage they need.

The utility of this tool was conceived and formulated to be color-coded and to be displayed in public view to ensure transparency in, and to facilitate, the distribution of patient assignment whose clear view assists in reassuring the nurses and PCTs that each of them has an unbiased and equitable share of the challenges confronting the unit.

The tool is also intended to be customizable based on the conditions peculiar to the hospital unit (e.g., existing number of acuity levels being used, the naming convention of pod/hallways and patient room identification, tasks that are unique to the hospital unit, deadline for all assessments to be completed, time at which a shift changes to the next, etc.)

### DETAILED DESCRIPTION OF THE INVENTION

The descriptions that follow are those based on the actual tool used during the clinical trial. Inasmuch as the appearance of this invention has been customized to the requirements of the hospital unit which it was used for, the materials, dimensions and colors shown herein should not be construed as defining and limiting the only appearance by which the invention may be constructed or presented.

### The Physical Board

The NPAT consists of grids of intersecting horizontal rows and vertical columns printed on a white glare-free laminated water-resistant sheet of paper as its top layer (FIG. 1), which is then underlain by a 19⅞-in×19⅞-in 16-gauge (GA) galvanized iron (GI) sheet. Underneath this GI-sheet is the bottom layer made of 19⅞-in×19⅞-in black illustration board as backing material for the frame (FIG. 2). The finished board is encased in a 20-in ×20-in×2-in pre-cut self-assembled black aluminum frame. The size can be customized to the headcount of the hospital unit and to the volume of summary information that the unit needs to monitor each shift.

The board is mounted on an easel stand (FIG. 2).

The laminated sheet of paper can be written on with figures such as census, date, number of Total Care patients, number of patients with Seizures, with Restraints, with Central Lines, etc. using permanent markers. Alcohol wipes can then be used to update the figures.

Patient details, as will be further discussed below, are printed on heavy-weight coated magnetized sheet, same grade as that attached to vehicles for advertising purposes, and cut to various shapes (e.g., circle, square, rectangle, etc.) and to sizes in colors depending on the volume of information that the hospital unit needs to monitor.

On each cut-out is printed either of the following patient details:

Patient Acuity, which is “the measure of a patient's severity of illness or medical conditions including, but not limited to, the stability of physiological and psychological parameters and the dependency needs of the patient and the patient's family.”⁴ Current convention assigns a higher acuity to patients with more severe conditions, or more dependent patient or family member. The acuity is color-coded to ensure that patients with high acuity are not concentrated to a few nurses and are instead equitably distributed across all nurses on-shift. The acuity levels depend on those set by the hospital. Hence, they are not limited to the example mentioned below, namely:
red for high acuity or complex workload.
yellow for moderate acuity or moderate workload; and
green for low acuity or low workload. ⁴ From www.lawinsider.com/dictionary/patient-acuity

Patient room number color-coded based on the room's hall/pod location. The color-coding helps in ensuring that a nurse is assigned to cover a maximum of two halls or pods.

Isolation precautions are color-coded and labelled with either of the following:
Airborne
Contact
Droplet
Enhanced airborne
Enteric
Neutropenic

Other customized indicators that the unit deems important to convey immediate information indicating need for close/constant monitoring or special treatment or care required for the patient as follows:
TC for Total Care
SZ for Seizure
RST for Restraints
ACHS/Q6H for blood sugar check before meals and at night or every 6 hours; and
POS DC for Possible Discharge

Unique nickname of each nurse and PCT on-shift who are assigned with a set of patients.

Inasmuch as the number of nurses is invariably more than that of PCTs on-shift, the set of patients assigned to a nurse is different from that assigned to a PCT.

A “check mark” sign alongside a nurse's nickname indicating completion of assessment of all patients assigned under said nurse.

A “question mark” sign alongside a nurse's nickname indicating incomplete or un-finished assessment of at least one patient assigned under said nurse

Blank cut-outs are provided for extra names of newly hired nurses or PCTs.

A room number not underlain with an acuity cut-out and assigned to a nurse indicates that the room is yet empty and the nurse should expect a possible patient admission.

These color-coded magnetized cut-outs are laid onto the laminated water-resistant grid-printed sheet underlain with the GI-sheet. The printed sheet consists of two grids.

The upper grid (FIG. 8) provides a space where the CN jots down the headcounts of patients requiring special care/attention or precautionary measures, and is labelled with the following column headers, viz:

| COLUMN HEADER | PURPOSE |
|---|---|
| DATE | Current date |
| SHIFT IDENTIFIER | AM for day shift; PM for night shift; may |
| | be modified depending on shift |
| | classification adopted by the hospital unit |
| CENSUS | Total number of patients under the care of |
| | the hospital unit |
| CENTRAL LINE (CL) | Number of patients with a central line |
| | attached |
| FOLEY | Number of patients with a foley attached |
| FALL PRECAUTION | Number of patients who are at-risk of |
| | what the hospital unit may classify as a |
| | “fall accident” |
| TOTAL CARE (TC) | Number of patients requiring total care |
| | (i.e., totally dependent on a caregiver) |
| DO-NOT-RESUSCITATE (DNR) | Number of patients who need not be |
| | resuscitated if manifesting signs of |
| | necrosis |
| POSSIBLE DISCHARGE (POS_DC) | Number of patients who may be released |
| | during the shift from the care of the |
| | hospital unit |
| ELOPEMENT PRECAUTION (EP) | Number of patients who have actual |
| | records, or exhibit the potential, of leaving |
| | the hospital unit's premises without |
| | proper authorization |
| RESTRAINTS (RST) | Number of patients who are at-risk of |
| | endangering themselves and are, |
| | therefore, currently administered with |
| | precautionary measures that limit the |
| | patient's mobility |
| PRECAUTION: AIRBORNE | Number of patients afflicted with illness |
| | transmissible by airflow |
| PRECAUTION: CONTACT | Number of patients afflicted with illness |
| | transmissible by touching any part of the |
| | patient's body or any object touched by |
| | the patient |
| PRECAUTION: DROPLET | Number of patients afflicted with illness |
| | transmissible by patient's breathing, |
| | talking, coughing or sneezing |
| PRECAUTION: ENTERIC | Number of patients afflicted with illness |
| | transmissible by touching waste from the |
| | patient's digestive system |
| PRECAUTION: ENHANCED | Number of patients afflicted with illness |
| AIRBORNE | transmissible by air, contact, droplets |
| | specifically COVID cases |
| PRECAUTION: NEUTRO | Number of patients with abnormally low |
| | levels of white blood cells and are, |
| | therefore, more prone to contract |
| | nosocomial infections |

The central grid (FIG. 9) contains the nurse-to-patient assignment itself arranged in the form of intersecting horizontal rows and vertical columns whereby a patient room number is lodged in each of these row-column intersections beginning from the third column from the left. Each row of patient room numbers is then assigned to a specific nurse who is identified in the second column from the left.

The first and last columns (FIG. 10) are set aside for informational flags identifying the status of the assessment of the patients assigned to each nurse. A question mark cut-out is laid by the CN on the rightmost column labelled “Pending Evaluation of Primary Nurses.” This procedure is done on all rows of patients at the start of the assessments for the shift.

Unused cut-outs are stored in the NPAT detail organizer box (FIG. 4, FIG. 5). The cover of the organizer shows a sample arrangement of how the magnetic cut-outs are laid on the NPAT board.

### The Software

The descriptions that follow are those based on the software version of the tool. The colors shown herein should not be construed as defining the only appearance by which the invention may be coded or presented.

The NPAT software (FIG. 13) is coded in HTML and CSS for front-end presentation, and PHP for backend computations. It uses MySQL as the database engine for data storage. It is compiled using ExeOutput for PHP as a 32-bit stand-alone executable program. The current version runs under a Windows 10 operating system on desktop computers only.

The software is menu-driven (FIG. 14) with dropdown lists. Data entry is either by mouse-click or keyboard.

The current version of the software is intended to be installed on-premises—i.e., on the desktop of the hospital unit rather than on a server or in the cloud.

CNs are granted administrator privileges who may add or remove nurses or PCTs from the list of authorized users.

CNs and nurses need to register themselves to be able to use the software. PCTs are registered by a CN.

Each NPAT software is customized to suit the conditions prevailing at, and requirements of, the hospital unit where it will be used. Such customization includes but may not be limited to existing number of acuity levels being used, the naming convention of pod/hallways and patient room identification, deadline for all assessments to be completed, time at which a shift changes to the next and other conditions unique to the hospital unit. Customization is being applied in every release of the software to avoid the unauthorized use of the same software version in other hospital units outside of the unit in which the software would have been licensed.

### How the NPAT Board Is Used

To attain an equitably distributed assignment of patients that will enable the nursing unit to adequately perform its function while ensuring the safety and promoting the well-being of both the nurses and the patients, the following steps need to be undertaken:
Assessment of patients by the nurses using a SPAAT
Assignment of patients to nurses and PCTs by the CN using the NPAT based on the output from the SPAAT

### Assessment Using the Physical SPAAT

During the maiden use of the NPAT, the current nurse-to-patient assignment is initially adopted. Succeeding nurse-to-patient assignments will then be based on the results of the SPAAT-based assessment and workload re-distribution using the NPAT.

The nurse assesses each patient according to the hospital unit's SPAAT. The use of a SPAAT is an EBP in nursing adopted with permission from the American Nurses Association.

The SPAAT is customized according to the patient population of the specific hospital unit where it is being applied (e.g., Neurology, Cardiology, OB-Gyne, Oncology, etc.) to keep each category of assessments relevant. The customized content is designed by this nurse-inventor based on the pre-application survey of all nurses in the hospital unit. The survey aimed at capturing any patient-condition and/or any assessment category relevant to the patient population that may not yet be included in this nurse-inventor's pro-forma SPAAT.

Areas of assessment can include but need not be limited to the following categories, to wit:
ADMISSION
ADLs (Activities of Daily Living)
PSYCHOSOCIAL/EDUCATION/BEHAVIORAL/COMMUNICATION
CARDIOLOGY
DIALYSIS
DISCHARGE PLAN
GASTROINTESTINAL
GENITOURINARY
INFUSION
INTEGUMENTARY
ISOLATION PRECAUTION
LEVEL OF CONSCIOUSNESS
MEDICATIONS
NEUROLOGY
PAIN CONTROL
PROCEDURES
RESPIRATORY
VITAL SIGNS
RESUSCITATION ORDERS

Each nurse goes about his/her round of assessments using the SPAAT.

In the physical board version of the NPAT, the SPAAT is in the form of a laminated sheet of legal-sized paper bearing the patient room number printed on the top right corner, and all the categories and the standardized assessment descriptions.

Each room has a designated SPAAT, which is kept in the patient server. Alternatively, depending on the hospital unit's preference, the SPAAT of each room is collected from a binder, which may be lodged at the nurses' station. FIG. 6 shows the actual SPAAT, used during the clinical test conducted in 2018 in the Neurology Unit of an acute hospital in Central Texas. This format of the SPAAT shows that it was for Room 38 and accessed via the Patient Server. The same SPAAT-form can be accessed via a book binder (signified by the three punched holes along the left margin (FIG. 7) of that for Room 33) kept at the nurses' station. Note the absence in both formats of any personal information other than the room number identifying the patient location.

In the SPAAT, each category consists of a set of standard descriptions of the status of a patient, which is prefixed with a bullet mark and falls under a specific column of acuity level.

Following current convention, the NPAT has been structured to classify each standardized description according to the following levels, to wit:

TABLE 2
Acuity Level Color and Description

| ACUITY LEVEL | COLOR | DESCRIPTION |
|---|---|---|
| 1 | Green | stable patient with low workload |
| 2 | Yellow | complex patient with moderate |
| | | workload |
| 3 | Red | high-risk patient with high |
| | | workload |

The nurse's assessment of the patient is recorded by manually ticking the bullet, either by a check or a cross mark, using a felt-tipped permanent marker whose ink can be erased using alcohol for later use by the next round of assessments by the nurses of the incoming shift.

Algorithm for Determining Patient Acuity—The acuity assigned to the patient is the highest acuity assessed on the patient across all the categories regardless of frequency or count.

After the nurse has finished the assessment of some or all of the patients under his/her care, she reports back to the nurses' station and updates the NPAT board attaching it with the appropriate cut-outs based on the result of his/her assessments.

Nurses are the only authorized personnel who, at any time during their shift, may update in the NPAT the acuity of the patients assigned to them.

FIG. 3 shows an actual filled-in NPAT board used during the clinical trial showing the color-coded magnetic cut-outs posted on a grid consisting of six maximum-patient-per-nurse vertical columns by seven nurses-on-shift horizontal rows. Patient acuities are indicated in large rectangular color-coded cut-outs. In this clinical test, the colors red, yellow and green were used for high, moderate and low acuities, respectively. Some of these acuity cut-outs are printed with specialized treatment needs (e.g., ACHS, QC and/or TC). Other specialized treatment needs (e.g., HRPU, RST, SZ) are printed on smaller white rectangular cut-outs. Room numbers are circumscribed in a circle printed on square cut-outs color-coded as to their hall/pod location. Isolation precautions are printed on small rectangular cut-outs whose color matches that of the five colored cells at the board's upper righthand corner just below the “NURSE-PATIENT ASSIGNMENT TOOL (NPAT)” board title. Along the header line below this board title are hand-written numbers indicating the shift information such as date, census, number of Total Care patients, number of patients with Seizures, with Restraints, and with Central Lines; and the number patients requiring certain isolation precautions to be observed when treating them. The first column from the left contains cut-outs for check marks indicating completion of assessment of all patients assigned to the nurse along that row. The second column indicates the hall/pod assignment of the nurse posted under the third column. The last column on the right contains cut-outs for question marks indicating assessment of all patients along that row with a question mark is not yet completed. The lower left quadrant of the board shows the hand-written numbers of patient rooms assigned to the PCTs (FIG. 11). The numbers circumscribed in a circle printed on small square cut-outs in this quadrant indicate the patient rooms requiring Total Care. A legend block is printed on the lower right quadrant of the NPAT board (FIG. 12).

The question mark cut-out at the rightmost column along the nurse's assigned row of patients is replaced by the nurse himself/herself with a check mark cut-out laid on the leftmost column alongside his/her name as soon as the assessment of all patients under his/her care is completed.

Possible Admissions are assigned with high acuity due to the time-consuming preparations of the room, patient assessment and the documentation involved during the admission process.

### Assignment of Patients to Nurses and PCTs Using the NPAT Board

The ultimate purpose of equitably distributing workload assignments will be defeated if the CN starts the distribution process when the assessment of some patients is still pending. Thus, it is imperative that the CN monitors the status of the NPAT board and regularly reminds nurses who are falling behind the target completion deadline for all assessments to ensure that all assessments are completed on-schedule. This deadline may vary across hospital units depending on the unit's shift schedule.

Regular monitoring by the CN of the status of assessments could also help him/her evaluate if there is a need for additional support or reduction in manpower or if the current staffing is just adequate.

The CN begins assigning patients to the nurses on-shift as soon as: 1) the NPAT board has been updated based on the results of the nurses' assessments; and 2) all check-mark cut-outs on all rows of the NPAT board have been attached indicating completion of all assessments. This is done by manually rearranging the largest rectangular color-coded magnetized cut-out for acuity attached with all other cut-outs for room number/hall location, specialized treatment care and isolation precautions while keeping in mind the following guidelines, namely:
To count a Possible Admission (POS_ADM) as a high-acuity patient in view of the time-consuming documentation of patient information and status upon admission, and preparation of patient room that the nurse and the PCT, respectively, must undertake. This NPAT invention facilitates re-assignment of POS_ADM patients by making available a “POS_ADM” cut-out for laying over the high-acuity cut-out attached with the patient room number.

Whenever permissible, to equally distribute, high acuity patients among all nurses on-shift. When the total number of high acuity patients is not an integral multiple of the number of nurses on-shift, the prerogative to assign a higher number of high acuity patients to only a certain number of nurses then rests on the CN on-shift. This NPAT invention facilitates the re-assignment of high acuities by color-coding high acuity patients.

Whenever permissible, to equally distribute, medium acuity patients among all nurses on-shift. When the total number of medium acuity patients is not an integral multiple of the number of nurses on-shift, the prerogative to assign a higher number of medium acuity patients to only a certain number of nurses then rests on the CN on-shift. This NPAT invention facilitates the re-assignment of medium acuities by color-coding medium acuity patients.

Whenever permissible, to equally distribute, low acuity patients among all nurses on-shift. When the total number of low acuity patients is not an integral multiple of the number of nurses on-shift, the prerogative to assign a higher number of low acuity patients to only a certain number of nurses then rests on the CN on-shift. This NPAT invention facilitates the re-assignment of low acuities by color-coding low acuity patients.

Whenever permissible, to assign a nurse, who is already assigned with a patient flagged with a Neutropenic isolation precaution, to no other patient flagged with any of the other isolation precautions. This NPAT invention facilitates the isolation of Neutropenic patients from other isolation patients by color-coding isolation precaution flags.

Whenever permissible, to assign a nurse to a maximum of two halls or pods—i.e., no three patients assigned to the same nurse should be in more than two separate halls or pods. This NPAT invention facilitates the limiting of hall/pod assignment to a certain maximum by color-coding patient room numbers based on the room's hall/pod location. Based on this nurse-inventor's own experience, coverage of no more than two halls/pods is manageable.

After distributing the workload assignment for nurses, the CN then assigns patients among PCTs on-shift. This is done by attaching on the lower-left quadrant of the board a second set of the cut-outs for room numbers specifically for PCTs, while keeping in mind the following guidelines:

Whenever permissible, to assign a PCT to a maximum of one hall or pod—i.e., no two patients assigned to the same PCT should be in more than one hall or pod. This NPAT invention facilitates the limiting of hall/pod assignment to a certain maximum by color-coding patient room numbers based on the room's hall/pod location. Based on this nurse-inventor's observation, coverage of no more than one hall/pod by a PCT is manageable.

Whenever permissible, to equally distribute, Total Care (TC) patients among all PCTs on-shift. When the total number of TC patients is not an integral multiple of the number of PCTs on-shift, the prerogative to assign a higher number of TC patients to only a certain number of PCTs then rests on the CN on-shift. This NPAT invention facilitates the re-assignment of TC patients by providing “TC” cut-outs for attachment on the room number cut-out if needed.

Inasmuch as nurses outnumber PCTs, the latter will invariably be working with more than one nurse.

This new nurse-to-patient and PCT-to-patient assignments, which was prepared based on the SPAAT-based assessments by the nurses of the current shift and on the NPAT-based workload re-distribution by the CN of the current shift, will then be turned-over to and used by the incoming shift. The incoming shift, once the turn-over has been completed, will then do its own round of 1) assessing patients using the SPAAT under each nurse's care and 2) updating the NPAT board based on such assessments. The new CN on-shift will then review the status of nurse-to-patient and PCT-to-patient assignments and, using the NPAT board, adjust them, if warranted. Thus, the cycle continues.

The physical NPAT board, after all adjustments have been made, is finally displayed at the nurses' station to facilitate dissemination to and viewing by all nurses and PCTs of their workload assignments.

### How the NPAT Software is Used

To attain a well-distributed assignment of patients that is fair and equitable and that will enable the nursing unit to adequately perform its function while ensuring the safety and promoting the well-being of both the nurses and the patients, the following steps need to be undertaken:
Assessment of patients by the nurses using the software version of the SPAAT
Assignment of patients to nurses and PCTs by the CN using the software version of the NPAT based on the output from the SPAAT

During the maiden use of the NPAT, the current nurse-to-patient assignment is initially adopted, which is entered into the NPAT software by selecting “Pre-Assign>Nurses” in the main menu (FIG. 15) where the CN defines the following:
Maximum Patients per Nurse
Total Nurses-on-Shift Today (including Charge Nurse)
Code Name of nurses on-shift

Similarly, for the PCTs during the maiden use of the NPAT, the current PCT-to-patient assignment is initially adopted, which is entered into the NPAT software by selecting “Pre-Assign>PCTs” in the main menu (FIG. 16). During the “pre-assignment” step, the CN defines the following:
Maximum Patients per PCT
Total PCTs-on-Shift Today
Code Name of PCTs on-shift

Succeeding PCT-to-patient assignments will then be based on the results of the SPAAT-based assessment and workload re-distribution using the NPAT.

### Assessment Using the SPAAT Module in NPAT

The nurse assesses each patient according to the hospital unit's SPAAT. The use of a SPAAT is an EBP in nursing adopted with permission from the American Nurses Association.

The SPAAT is customized according to the patient population of the specific hospital unit where it is being applied (e.g., Neurology, Cardiology, OB-Gyne, Oncology, etc.) to keep each category of assessments relevant. The customized content is designed by this nurse-inventor based on the pre-application survey of all nurses in the hospital unit. The survey aimed at capturing any patient-condition and/or any assessment category relevant to the patient population that may not yet be included in this nurse-inventor's pro-forma SPAAT.

Areas of assessment can include but need not be limited to the following categories, to wit:
ADMISSION
ADLs (Activities of Daily Living)
PSYCHOSOCIAL/EDUCATION/BEHAVIORAL/COMMUNICATION
CARDIOLOGY
DIALYSIS
DISCHARGE PLAN
GASTROINTESTINAL
GENITOURINARY
INFUSION
INTEGUMENTARY
ISOLATION PRECAUTION
LEVEL OF CONSCIOUSNESS
MEDICATIONS
NEUROLOGY
PAIN CONTROL
PROCEDURES
RESPIRATORY
VITAL SIGNS
RESUSCITATION ORDERS

Each nurse goes about his/her round of assessments using the physical SPAAT designated for each patient. This is accessible either via the patient server or in the SPAAT binder located at the nurses' station.

Once the nurse has finished the assessment of some or all of the patients under his/her care, she reports back to the nurses' station and logs in the NPAT software and enters his/her assessment for each patient using the software's SPAAT module by selecting “Assess Patients” in the main menu (FIG. 17). The software will then provide dialogue boxes (FIG. 18 and FIG. 19) that will guide the nurse until he/she gets into the SPAAT pages themselves.

The SPAAT module consists of several pages, each of which covers one of the categories listed in Paragraph above. Each category consists of a set of standardized description of the status of a patient, which is prefixed with a radio button and falls under a specific column of acuity level (FIG. 20).

The nurse's assessment is recorded by selecting a radio button with the mouse.

Some of the descriptions in a set may not be exhibited by, or not manifest in, the patient or may even be mutually exclusive—i.e., if one condition is true, the rest of the conditions cannot possibly and simultaneously be true. In the NPAT board version, the exclusion of conditions in a set of mutually exclusive descriptions or when none of the conditions is true is an error-prone manual task that the assessing nurse must vigilantly take note of by ensuring that the appropriate bullet point, or none whichever is appropriate, is ticked off in the set. In the software version, this exclusion is automatically made possible by the radio button—i.e., if a radio button is selected, the rest of the radio buttons for the other descriptions are automatically deselected. Also in the software version, a fourth column labelled “None Applies” allows deselection of all descriptions in a set (FIG. 20).

Following current convention, the NPAT has been structured to classify each standardized description according to the following levels, to wit:

### Table 3—Acuity Level Color and Description

TABLE 3
Acuity Level Color and Description

| ACUITY LEVEL | COLOR | DESCRIPTION |
|---|---|---|
| 1 | Green | stable patient with low workload |
| 2 | Yellow | complex patient with moderate workload |
| 3 | Red | high-risk patient with high workload |

Algorithm for Determining Patient Acuity—The acuity assigned to the patient is the highest acuity assessed on the patient across all the categories regardless of frequency or count.

Nurses are the only authorized personnel who, at any time during their shift, may update in the NPAT the acuity of the patients assigned to them by updating the SPAAT module of the NPAT software.

Every update of the SPAAT module is immediately reflected on the NPAT grid of the software by invoking “Tabulate Assessment> and Nurse Assignment> in the main menu (FIG. 21). However, the nurse may opt to complete updating the SPAAT module for all patients under his/her care before displaying the results on the NPAT grid or table.

FIG. 22 shows an updated NPAT grid of nurse-to-patient assignment showing the color-coded cells posted on a grid consisting of five maximum-patient-per-nurse vertical columns by five nurses-on-shift horizontal rows. Patient acuities are indicated in large rectangular color-coded cells. In this screenshot, the colors red, yellow and green were used as background to indicate patients assessed with high, moderate and low acuities, respectively. Some of these cells include notes for specialized treatment needs (e.g., ACHS, QC and/or TC, HRPU, RST, SZ, etc.) that appear on the left side of the room numbers (FIG. 23). Room numbers, inscribed in rectangular cells, are color-coded as to their hall/pod location. Isolation precautions appear as boxed notes on the righthand side (FIG. 24) whose colors match those of the six colored cells at the board's upper righthand corner just below the “NURSE-PATIENT ASSIGNMENT TOOL (NPAT)” table title (FIG. 25). Along the header line below this table title are figures indicating shift information such as date, census, number of patients with Central Line, Foley, Fall Precaution, on Total Care, on Do-Not-Resuscitate, for Possible Discharge, on Elopement Precaution or with Restraints; and the number of patients requiring certain isolation precautions to be observed when treating them (FIG. 26). The first column from the left displays icons for either a white check mark inscribed in a green circle indicating completion of assessment of all patients assigned to the nurse along that row or a white exclamation point inscribed in a red triangle indicating incomplete or pending assessment of at least one patient assigned to the nurse along that row (FIG. 27). This column is automatically updated by the software every time an assessment is completed. The second column lists the nurses assigned to the row of patients to the right (FIG. 28). Room numbers of patients who have not yet been completely assessed are underlined (FIG. 29).

FIG. 30 shows an updated NPAT grid of PCT-to patient assignment showing the color-coded cells posted on a grid consisting of eight maximum-patient-per-PCT vertical columns by three PCTs-on-shift horizontal rows.

Possible Admissions are assigned with high acuity due to the time-consuming preparations of the room and the documentations involved during the admission process. The room number is overlain with a watermark labelled “POSSIBLE ADMISSION” diagonally across the room number (FIG. 31).

### Assignment of Patients to Nurses and PCTs Using the NPAT Software

The ultimate purpose of equitably distributing workload assignments will be defeated if the CN starts the distribution process when the assessment of some patients is still pending. Thus, it is imperative that the CN monitors the status of the NPAT board and regularly reminds nurses who are falling behind the target completion deadline for all assessments, to ensure that all assessments are completed on-schedule. This deadline may vary across hospital units depending on the unit's shift schedule (FIG. 32).

Regular monitoring by the CN of the status of assessments could also help him/her evaluate if there is a need for additional support or reduction in manpower or if the current staffing is just adequate.

Completion of all assessments is indicated when:
All exclamation points in the first column had been replaced with check marks.

No patient room is underlined.

When all assessments have been completed, the CN begins assigning patients to the nurses on-shift. This is done by selecting “Tabulate_Assessment> and Nurse Assignment” in the main menu (FIG. 21) to display the status of the workload distribution.

If there is a need to re-assign a specific patient from one nurse to another, then the CN selects “Tweak>Nurse Assignment” in the main menu (FIG. 33).

If the tweak involves re-assignment of only one patient, then the CN/user selects using the mouse the room number to be re-assigned and selects next an un-occupied slot of the nurse whom the patient needs to be re-assigned to.

If the tweak involves a swap of two patients between two nurses, then the CN/user selects using the mouse the first room number to be re-assigned and selects next the second room number to be re-assigned.

The re-distribution of workload assignment or tweaking is done while keeping in mind the following guidelines:
To count a Possible Admission (POS_ADM) as a high-acuity patient in view of the time-consuming documentation of patient information and status upon admission, and preparation of patient room that the nurse and the PCT, respectively, must undertake. The software version of this NPAT invention facilitates re-assignment of POS_ADM patients by overlaying a patient room number with a watermark labelled “POSSIBLE ADMISSION.”

Whenever permissible, to equally distribute, high acuity patients among all nurses on-shift. When the total number of high acuity patients is not an integral multiple of the number of nurses on-shift, the prerogative to assign a higher number of high acuity patients to only a certain number of nurses then rests on the CN on-shift. This NPAT invention facilitates the re-assignment of high acuities by color-coding high acuity patients.

Whenever permissible, to equally distribute, medium acuity patients among all nurses on-shift. When the total number of medium acuity patients is not an integral multiple of the number of nurses on-shift, the prerogative to assign a higher number of medium acuity patients to only a certain number of nurses then rests on the CN on-shift. This NPAT invention facilitates the re-assignment of medium acuities by color-coding medium acuity patients.

Whenever permissible, to equally distribute, low acuity patients among all nurses on-shift. When the total number of low acuity patients is not an integral multiple of the number of nurses on-shift, the prerogative to assign a higher number of low acuity patients to only a certain number of nurses then rests on the CN on-shift. This NPAT invention facilitates the re-assignment of low acuities by color-coding low acuity patients.

Whenever permissible, to assign a nurse, who is already assigned with a patient flagged with a Neutropenic isolation precaution, to no other patient flagged with any of the other isolation precautions. The software version of this NPAT invention facilitates the isolation of Neutropenic patients from other isolation patients by displaying a color-coded “NEUTRO” isolation sub-cell near the bottom-right corner of the patient room number.

Whenever permissible, to assign a nurse to a maximum of two halls or pods—i.e., no three patients assigned to the same nurse should be in more than two separate halls or pods. This NPAT invention facilitates the limiting of hall/pod assignment to a certain maximum by color-coding patient room numbers based on the room's hall/pod location. Based on this nurse-inventor's own experience, coverage of no more than two halls/pods is manageable.

Whenever permissible, to assign a nurse with a maximum of two patients requiring one-to-one feeding (1:1-patients). The software version of this NPAT invention facilitates the limiting of 1:1-patients to a certain maximum by providing this count on the first column of the NPAT board alongside each nurse's name (FIG. 34).

After distributing the workload assignment for nurses, the CN then assigns patients among PCTs on-shift in the same fashion as with nurses. This is done via the Tweaking function while keeping in mind the following guidelines, namely:

Whenever permissible, to assign a PCT to a maximum of one hall or pod—i.e., no two patients assigned to the same PCT should be in more than one hall or pod. This NPAT invention facilitates the limiting of hall/pod assignment to a certain maximum by color-coding patient room numbers based on the room's hall/pod location. Based on this nurse-inventor's observation, coverage of no more than one hall/pod by a PCT is manageable.

Whenever permissible, to equally distribute, Total Care (TC) patients among all PCTs on-shift. When the total number of TC patients is not an integral multiple of the number of PCTs on-shift, the prerogative to assign a higher number of TC patients to only a certain number of PCTs then rests on the CN on-shift. The software version of this NPAT invention facilitates the re-assignment of TC patients by displaying a “TC” notation on the lefthand side of the room number of patients under Total Care (FIG. 35).

Inasmuch as nurses outnumber PCTs, the latter will invariably be working with more than one nurse.

This new nurse-to-patient and PCT-to-patient assignments, which were prepared based on the SPAAT-based assessments by the nurses of the current shift and on the NPAT-based workload re-distribution by the CN of the current shift, will then be turned-over to and used by the incoming shift. The incoming shift, once the turn-over has been completed, will then do its own round of 1) assessing patients using the SPAAT under each nurse's care and 2) updating the NPAT board based on such assessments. The new CN on-shift will then review the status of nurse-to-patient and PCT-to-patient assignments and, using the NPAT board, adjust them, if warranted. Thus, the cycle continues.

The NPAT grid or table of this invention's software version, after all adjustments have been made, is finally displayed on the desktop monitor at the nurses' station where the software is installed to facilitate dissemination to and viewing by all nurses and PCTs of their workload assignments. If budget of the hospital unit allows, the NPAT table may also be simultaneously broadcast via a private network through tv monitors hung at strategic points of the hospital unit or along its hallways.

The NPAT, therefore, enables:
assessment of the patient based on the EBP of using a Standard Patient Acuity Assessment Tool (SPAAT),
capturing of the nurse's SPAAT-based assessment,
objective determination of the patient's acuity,
color-coded “encapsulation” of the information pertaining to patient's acuity, room number and hall/pod location and specialized treatment requirements as well as the required contact precautions that attending nurses and PCTs need to observe,
display of this “encapsulated information” in the form of a grid of horizontal rows and vertical columns of nurse-to-patient and PCT-to-patient assignments,
manual re-arrangement of these assignments until an equitable distribution of challenging, moderate and easy patients is achieved while observing exceptions or limitations that the hospital unit would like to adopt, and efficient yet equitable distribution of workloads.

### CONCLUSION

Based on the favorable findings in the research commissioned by this patent applicant and completed on 2018 Dec. 5 by Thomas Frost, P. A., whereby he opined that “protection in the form of a utility patent may be available directed to the specific novel structural or functional features of your invention.” (refer to Atty. Frost's “International Patentability Search and Opinion (PPSO) on pages 1-2), this application for a non-provisional utility patent is herewith being submitted.

### CITATIONS

Acar I, Butt S E. Modeling Nurse-to-Patient assignments considering patient acuity and travel distance metrics. J Biomed Inform. 2016 December; 64:192-206. doi: 10.1016/j.jbi.2016.10.006. Epub 2016 Oct. 11. PMID: 27742350.
American Nurses Association (2018). Nurse fatigue. Retrieved from https://www.nursingworld.org/practice-policy/work-environment/health-safety/healthy-nurse-healthy-nation/nurse-fatigue/Chiulli K A, Thompson J, Reguin-Hartman K L. Development and implementation of a patient acuity tool for a medical-surgical unit. Academy of Medical-Surgical Nurses. 2014; 23 (2): 1, 9-12. Copyright® 2020. Academy of Medical-Surgical Nurses (AMSN). Used with permission. All rights reserved. AMSN.org.
DiClemente, Kimberly. 2018 Standardizing Patient Acuity: A Project on a Medical-Surgical/Cancer Care Unit. The Free Library (November 1), https://www.thefreelibrary.com/Standardizing Patient Acuity: A Project on a Medical-Surgical/Cancer . . . -a0568974186
Kidd M, Grove K, Kaiser M, Swoboda B, Taylor A. A new patient-acuity tool promotes equitable Nurse-to-Patient assignments. American Nurse Today. 2014; 9 (3): 1-4. Copyright®2019. HealthCom Media. Used with permission. All rights reserved. AmericanNurseToday.com

1. We claim that the npat is the first-of-its-kind tool for distributing nurse-to-patient assignments that leverages the color-coding method of displaying a patient's status comprising his/her acuity, room number, hall/pod location and specialized treatment needs as well as the isolation precautions that nurses and pcts need to observe when attending to the patient.

2. We claim that the NPAT is the first-of-its-kind tool for distributing nurse-to-patient assignments that gives a bird's-eye view of the distribution of patient acuity across halls, nurses and PCTs for the shift.

3. We claim that the NPAT is the first-of-its-kind tool for distributing nurse-to-patient assignments that is available in the form of 1) a physical board or 2) a computer software.
